(19) United States
(12) Patent Application Publication
(10) Pub. No.: US 2022/0336086 A1
(43) Pub. Date: Oct. 20, 2022

(54) METHOD AND SYSTEM FOR CAPTURING CATEGORIZED NOTES ON AN ULTRASOUND SYSTEM

(71) Applicant: GE Precision Healthcare LLC, Wauwatosa, WI (US)

(72) Inventors: Jenny Pagano, St. Georgen im Attergau (AT); Martin Swoboda, Hohenzell (AT)

(21) Appl. No.: 17/232,506

(22) Filed: Apr. 16, 2021

(51) Int. Cl. G16H 40/20; G06F 3/041; G06F 3/14; G06F 40/169; G16H 10/60; G16H 30/20; G16H 30/40; G06F 21/62; A61B 8/00

(52) U.S. Cl. CPC G16H 40/20; G06F 3/041; G06F 3/14; G06F 40/169; G16H 10/60; G16H 30/20; G16H 30/40; G06F 21/6245; A61B 8/468; A61B 8/463

(57) ABSTRACT

A system and method for capturing categorized notes on an ultrasound system is provided. The method includes receiving, by at least one processor of an ultrasound system executing a notes application, a selection to create a notes application document, presenting the notes application document at a display system, and inserting received notes in the notes application document. The method includes assigning an ultrasound topic category and/or an access privilege setting to the notes application document and selectively storing the notes application document having the received notes at one of a plurality of data storage media communicatively coupled to the ultrasound system based on the ultrasound topic category and/or the access privilege setting. The method includes retrieving the notes application document having the received notes from the one of the plurality of data storage media and presenting the notes application document having the received notes at the display system.

### FIELD

Certain embodiments relate to ultrasound systems. More specifically, certain embodiments relate to a method and system for capturing categorized notes on an ultrasound system. The categorized notes are separate and distinct from annotations embedded in specific ultrasound images of an ultrasound examination.

### BACKGROUND

Ultrasound imaging is a medical imaging technique for imaging organs and soft tissues in a human body. Ultrasound imaging uses real time, non-invasive high frequency sound waves to produce two-dimensional (2D), three-dimensional (3D), and/or four-dimensional (4D) (i.e., real-time/continuous 3D images) images.

Ultrasound imaging is a valuable, non-invasive tool for diagnosing various medical conditions. An ultrasound operator performing an ultrasound examination may analyze the acquired ultrasound image data to perform measurements and add annotations to the ultrasound images. However, an ultrasound operator may desire to record notes not related to a particular ultrasound image, but instead to an ultrasound-related topic, such as notes related to a particular patient, the ultrasound machine, the ultrasound operator, or an ultrasound examination generally, among other things.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for capturing categorized notes on an ultrasound system, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to capture categorized notes, in accordance with various embodiments.

FIG. 2 is a flow chart illustrating exemplary steps that may be utilized for capturing categorized notes on an ultrasound system, in accordance with exemplary embodiments.

### DETAILED DESCRIPTION

Certain embodiments may be found in a method and system for capturing categorized notes on an ultrasound system. Various embodiments have the technical effect of creating notes at an ultrasound system that are not embedded as annotations in an ultrasound image. Aspects of the present disclosure have the technical effect of categorizing created notes based on an ultrasound topic, such as the ultrasound machine, the ultrasound operator, an ultrasound patient, an ultrasound examination, or the like. Certain embodiments have the technical effect of allowing an ultrasound operator to view and create notes at a main display and/or at a touch panel display if the notes are private, for example. Various embodiments have the technical effect of providing a reminder time for presenting a notification at an ultrasound machine to review the created note. Aspects of the present disclosure have the technical effect of providing a dedicated area at a display system for accessing notes, entering notes, and/or receiving notifications related to notes. Certain embodiments have the technical effect of categorizing notes based on access privilege settings, such as access available to only a creator of the note, only to a group of selected people or a selected class of people, only to people with access to a categorized ultrasound topic, and/or only to users of a particular ultrasound machine. Various embodiments have the technical effect of selectively storing the created notes based on a categorized ultrasound topic and/or access privilege settings.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

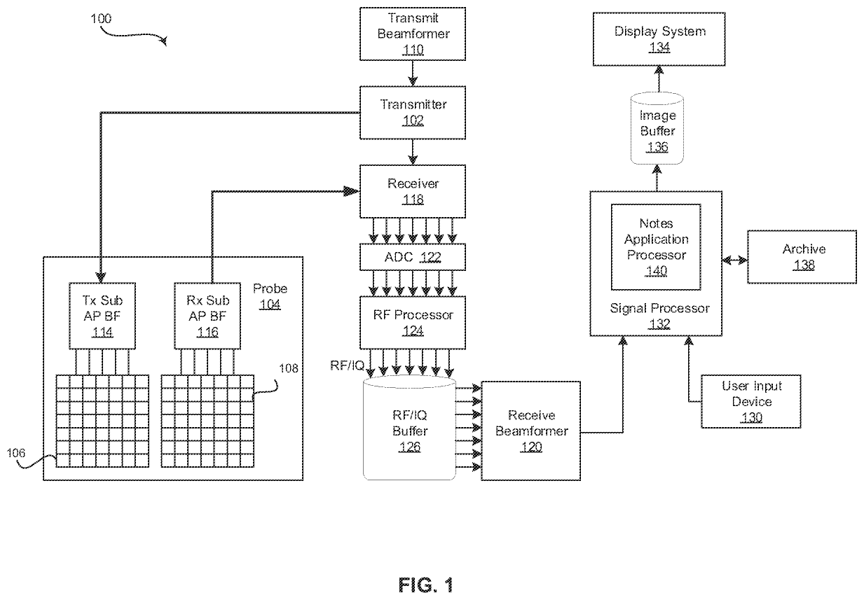

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to capture categorized notes, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as the heart, a blood vessel, a fetus, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, launch a notes application, enter notes, provide category and access privilege setting selections, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, a touch pad, a trackball, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), an enterprise archive (EA), a vendor-neutral archive (VNA), electronic medical records (EMR), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a notes application processor 140. The signal processor 132 may be capable of receiving input information from a user input device 130 and/or archive 138, receiving image data, generating an output displayable by a display system 134, and manipulating the output in response to input information from a user input device 130, among other things. The signal processor 132, including the notes application processor 140, may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a notes application processor 140 that comprises suitable logic, circuitry, interfaces and/or code that may be operable create notes application documents, categorize notes application documents, assign access privileges to notes application documents, store notes application documents, provide access to notes application documents, and provide notification regarding notes application documents by executing a notes application and responsive to user inputs. For example, the notes application processor 140 may be configured to automatically open a notes application at an ultrasound system 100 when the ultrasound system 100 is powered on or manually open in response to a user instruction provided via the user input device 130. In various embodiments, the notes application may be assigned a dedicated area of the display system 134, such as at one or more of at a main display or a secondary display (e.g., touch panel display). The notes application processor 140 may present selectable options and/or notifications in the dedicated area of the display system 134. For example, the selectable options may include a menu bar, a drop-down menu, selectable buttons, selectable icons, symbols, text, or the like. The notifications may include icons, symbols, text, or the like. In an exemplary embodiment, the notifications may be selectable to open a notes application document associated with the notification or to provide notification messages associated with one or more selectable notes application documents.

In a representative embodiment, the selectable options may include an option for creating a new notes document. For example, the notes application processor 140 may open and present a new notes application document in response to a user selection of a button, icon, drop-down menu item, or the like. The new notes application document may be presented in the dedicated area of the display system 134. In various embodiments, a user may configure a display location of the new notes application document. For example, the user may desire to view and/or enter notes into a document presented at a main display of the display system 134 for increased visibility. As another example, the user may desire to view and/or enter notes into a notes application document presented at a secondary display of the display system 134 that may be hidden from view of a patient or other people in an ultrasound examination room. The notes application processor 140 may receive and enter notes in the notes application document from an ultrasound operator via the user input device 130, such as a keyboard, touchscreen display, mousing device, speech recognition device, or any suitable user input device. The notes entered into the notes application document by the notes application processor 140 may be presented in real-time at the display system 134.

In an exemplary embodiment, the notes application processor 140 may be configured to categorize the notes application document in response to user selections via the user input device 130. For example, the notes application document may be categorized by ultrasound topics, such as the ultrasound machine, the ultrasound operator, an ultrasound patient, an ultrasound examination, or the like. As an example, a notes application document having notes regarding a next time to service an ultrasound machine, a missing or defective ultrasound probe 104 of the ultrasound system, or any suitable notes related to the particular ultrasound system 100 may be categorized under ultrasound machine notes. As another example, notes related to a schedule of an ultrasound operator, such as to move to a different ultrasound examination room at 2:00 PM, may be categorized under ultrasound operator notes. Notes related to a patient, such as having a reaction to a contrast agent, the patient having a husband that passed away last year, the patient having to wait 2 hours at a last appointment, or a patient not liking a particular doctor, may be categorized under ultrasound patient notes. Notes related to the difficulty in acquiring a particular ultrasound image view or any suitable notes generally related to an ultrasound examination but not specific to a particular acquired image, for example, may be categorized under ultrasound examination notes. In various embodiments, the ultrasound topic categorized may be pre-defined and may be selectable by the ultrasound operator creating the notes application document via drop-down selectable options, selectable buttons, selectable text, or the like. Additionally and/or alternatively, the notes application may be configured to allow an ultrasound operator to define a category or an additional category. In certain embodiments, the ultrasound topic categories may be associated with a storage location, such as a local archive, EMR, PACS, or any suitable data storage medium location. For example, ultrasound machine notes may be stored locally at the ultrasound system 100, ultrasound patient notes may be stored at EMR, and ultrasound examination notes may be stored at PACS, or any suitable storage associations.

In various embodiments, the notes application processor 140 may be configured to assign access privileges to a notes application document in response to user selections via the user input device 130. For example, the notes application document may be assigned access privileges such as access available to only a creator of the note, only to a group of selected people or a selected class of people, only to people with access to a categorized ultrasound topic, only to users of a particular ultrasound machine, and/or any suitable access privileges. In various embodiments, the access privileges may be pre-defined and may be selectable by the ultrasound operator creating the notes application document via drop-down selectable options, selectable buttons, selectable text, or the like. Additionally and/or alternatively, the notes application may be configured to allow an ultrasound operator to define access privileges. In certain embodiments, the access privileges may be associated with a storage location, such as a local archive, EMR, PACS, or any suitable data storage medium location. For example, access privileges assigning a notes application document only to users of a particular ultrasound machine may be stored locally at the particular ultrasound machine 100.

In certain embodiments, the notes application processor 140 may be configured to assign a reminder time for presenting a notification at an ultrasound machine to review the created note. For example, the reminder time may be assigned in response to a user selection via the user input device 130. As an example, the ultrasound operator that created a note to service the ultrasound system may assign a date and time for the notes application processor 140 to present a notification of the notes application document at the display system 134. As another example, the ultrasound operator that created a note to change ultrasound examination rooms at 2:00 PM may assign a date and time for the notes application processor to present a notification of the notes application document at the display system 134. The notification may be an icon, such as a red flag or exclamation point, a shape, a text message, a pop-up message of the notes application document, or any suitable notification.

In a representative embodiment, the notes application processor 140 may be configured to selectively store the created notes application document based on a categorized ultrasound topic and/or access privilege settings. For example, the notes application document may be associated with a storage location, such as a local archive, EMR, PACS, or any suitable data storage medium location based on the categorized ultrasound topic and/or access privilege settings. As an example, a notes application document categorized as ultrasound machine notes and/or assigned access privileges of only to users of a particular ultrasound machine may be stored locally at the particular ultrasound machine 100. As another example, a notes application document categorized as ultrasound patient notes may be stored at EMR. Additionally, a notes application document categorized as ultrasound examination notes may be stored at PACS.

In an exemplary embodiment, the notes application processor 140 may be configured to retrieve and present the notes application document at the display system 134 of the ultrasound system 100. The notes application processor 140 may retrieve and present the notes application document automatically and/or in response to an instruction received via a user input device 130. For example, the notes application processor 140 may automatically retrieve and present the notes application document based on a reminder time assigned to a notes application document. As another example, the notes application document processor 140 may retrieve and present the notes application document based on a selection of the notes application document or a notification associated with the notes application document. The notes application processor 140 may be configured to present the notes application document retrieved from the archive 138 or any suitable data storage medium at a dedicated area of a main display and/or touch panel display of the display system 134 of the ultrasound system 100.

Still referring to FIG. 1, the display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as a notes document, selectable notes categories, selectable notes access privilege settings, selectable notes notification times, notes notifications, and/or any suitable information. The display system 134 may include one or multiple displays. For example, the display system 134 may include a main display and a touch panel display, among other things. The display system 134 may be operable to provide a dedicated display area for accessing notes application documents, entering notes application documents, reviewing notes application documents, and/or receiving notifications related to notes application documents.

The archive 138 may be one or more computer-readable memories integrated with the ultrasound system 100 and/or communicatively coupled (e.g., over a network) to the ultrasound system 100, such as a Picture Archiving and Communication System (PACS), an enterprise archive (EA), a vendor-neutral archive (VNA), electronic medical records (EMR), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores a notes application, created notes application documents categorized by ultrasound topic and/or access privilege settings, notes category options, notes access privilege options, notes storage instructions, notes notification time options, notes notification display instructions, and/or notes document display instructions, among other things.

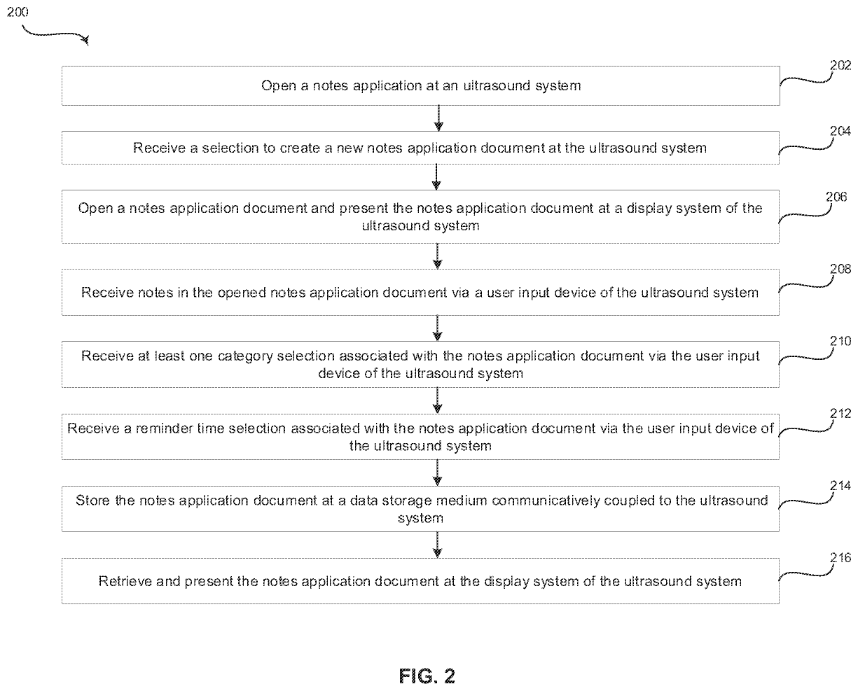

FIG. 2 is a flow chart 200 illustrating exemplary steps 202-216 that may be utilized for capturing categorized notes on an ultrasound system 100, in accordance with exemplary embodiments. Referring to FIG. 2, there is shown a flow chart 200 comprising exemplary steps 202 through 216. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 202, a notes application may be opened at an ultrasound system 100. For example, a notes application processor 140 of a signal processor 132 of the ultrasound system 100 may be configured to automatically open a notes application at an ultrasound system 100 when the ultrasound system 100 is powered on. As another example, the notes application processor 140 may open the notes application in response to a user instruction provided via the user input device 130. The notes application may be assigned a dedicated area of the display system 134, such as at one or more of at a main display or a secondary display (e.g., touch panel display).

At step 204, the signal processor 132 of the ultrasound system 100 may receive a selection to create a new notes application document at the ultrasound system 100. For example, the notes application processor 140 of the signal processor 132 may present selectable options in the dedicated area of the display system 134. The selectable options may include a menu bar, a drop-down menu, selectable buttons, selectable icons, symbols, text, or the like. The selectable options may include an option for creating a new notes document.

At step 206, the signal processor 132 may open a notes application document and present the notes application document at a display system 134 of the ultrasound system 100. For example, the notes application processor 140 of the signal processor 132 may open and present a new notes application document in response to a user selection at step 204. The notes application processor 140 may be configured to present the new notes application document in the dedicated area of the display system 134. In various embodiments, the display size, location, and/or the like may be user configurable. For example, the user may desire to view and/or enter notes into a document presented at a main display of the display system 134 for increased visibility or at a touch panel display for increased privacy.

At step 208, the signal processor 132 may receive notes in the opened notes application document via a user input device 130 of the ultrasound system 100. For example, the notes application processor 140 of the signal processor 132 may receive and enter notes in the notes application document from an ultrasound operator via the user input device 130, such as a keyboard, touchscreen display, mousing device, speech recognition device, or any suitable user input device. The notes entered into the notes application document by the notes application processor 140 may be presented in real-time at the display system 134.

At step 210, the signal processor 132 may receive at least one category selection associated with the notes application document via the user input device 130 of the ultrasound system 100. For example, the notes application processor 140 of the signal processor may be configured to categorize the notes application document in response to user selections via the user input device 130. The notes application document may be categorized by ultrasound topics, such as the ultrasound machine, the ultrasound operator, an ultrasound patient, an ultrasound examination, or the like. Additionally and/or alternatively, the notes application may be categorized by assign access privileges to a notes application document in response to user selections via the user input device 130. For example, the notes application document may be assigned access privileges such as access available to only a creator of the note, only to a group of selected people or a selected class of people, only to people with access to a categorized ultrasound topic, only to users of a particular ultrasound machine, and/or any suitable access privileges. In various embodiments, the ultrasound topic categories and/or access privilege settings may be pre-defined and may be selectable by the ultrasound operator creating the notes application document via drop-down selectable options, selectable buttons, selectable text, or the like. Additionally and/or alternatively, the notes application may be configured to allow an ultrasound operator to define a category, an additional category, and/or access privileges.

At step 212, the signal processor 132 may receive a reminder time selection associated with the notes application document via the user input device 130 of the ultrasound system 100. For example, the notes application processor 140 of the signal processor 132 may be configured to assign a reminder time for presenting a notification at an ultrasound machine to review the created note. The reminder time may be assigned in response to a user selection via the user input device 130. The reminder time may include a date and a time for presenting a notification and/or the notes application document at the display system 134 of the ultrasound system 100.

At step 214, the signal processor 132 may store the notes application document at a data storage medium 138 communicatively coupled to the ultrasound system 100. For example, the notes application processor 140 of the signal processor 132 may be configured to selectively store the created notes application document based on a categorized ultrasound topic and/or access privilege settings. The notes application document may be associated with a storage location, such as a local archive, EMR, PACS, or any suitable data storage medium location based on the categorized ultrasound topic and/or access privilege settings. As an example, a notes application document categorized as ultrasound machine notes and/or assigned access privileges of only to users of a particular ultrasound machine may be stored locally at the particular ultrasound machine 100. As another example, a notes application document categorized as ultrasound patient notes may be stored at EMR. Additionally, a notes application document categorized as ultrasound examination notes may be stored at PACS.

At step 216, the signal processor 132 may retrieve and present the notes application document at the display system 134 of the ultrasound system 100. For example, the notes application processor 140 of the signal processor 132 may be configured to retrieve and present the notes application document at the display system 134 of the ultrasound system 100 automatically and/or in response to an instruction received via a user input device 130. As an example, the notes application processor 140 may automatically retrieve and present the notes application document based on a reminder time assigned to a notes application document. As another example, the notes application document processor 140 may retrieve and present the notes application document based on a selection of the notes application document or a notification associated with the notes application document. The notes application processor 140 may be configured to present the notes application document retrieved from the archive 138 or any suitable data storage medium at a dedicated area of a main display and/or touch panel display of the display system 134 of the ultrasound system 100.

In various embodiments, the notes application may be implemented on a workstation, such as a workstation in a healthcare environment configured to review ultrasound examinations. A signal processor of the workstation may be configured to execute the notes applications as discussed above with respect to the ultrasound system 100. The signal processor of the workstation may be configured to create and review notes application documents associated with a particular ultrasound examination, separate and distinct from annotations of particular images of the ultrasound examination. The created notes may be categorized as associated with the particular ultrasound examination and stored in association with the particular ultrasound examination. The signal processor of the workstation may be configured to assign access privileges and/or a reminder time to a created notes application document. The signal processor may be configured to retrieve and present notes application documents at a display system of the workstation based on the ultrasound topic categories, access privileges and/or reminder times. The signal processor may be configured to create and review notes application documents associated with other ultrasound topic categories, such as a particular ultrasound machine, a particular ultrasound operator or other medical personnel, a particular ultrasound patient, or the like. The notes created at the workstation may be accessible at the workstation or the ultrasound system 100 based on the access privilege settings. The signal processor, display system, archive, and user input devices of the workstation may share various characteristics with the signal processor 132, notes application processor 140, display system 134, archive 138, and user input devices 130 of the ultrasound system 100 as described above in connection with FIGS. 1 and 2.

Aspects of the present disclosure provide a system and method 200 for capturing categorized notes on an ultrasound system 100. In accordance with various embodiments, the method 200 may comprise receiving 204, by at least one processor 132, 140 of an ultrasound system 100 executing a notes application, a selection to create a notes application document. The method 200 may comprise presenting 206, by the at least one processor 132, 140 via a display system 134 of the ultrasound system 100, the notes application document. The method 200 may comprise inserting 208, by the at least one processor, received notes in the notes application document presented at the display system 134. The method 200 may comprise assigning 210, by the at least one processor 132, 140, one or both of an ultrasound topic category and an access privilege setting to the notes application document. The method 200 may comprise selectively storing 214, by the at least one processor 132, 140, the notes application document having the received notes at one of a plurality of data storage media 138 communicatively coupled to the ultrasound system 100 based on the one or both of the ultrasound topic category and the access privilege setting assigned to the notes application document. The method 200 may comprise retrieving 216, by the at least one processor 132, 140, the notes application document having the received notes from the one of the plurality of data storage media 138. The method 200 may comprise presenting 216, by the at least one processor 132 140, the notes application document having the received notes at the display system 134.

In a representative embodiment, the received notes inserted in the notes application document are separate and distinct from annotations embedded in an ultrasound image of an ultrasound examination. In an exemplary embodiment, the ultrasound topic category is one of a plurality of selectable ultrasound topic categories comprising an ultrasound machine 100, an ultrasound operator, a patient, and/or an ultrasound examination. In various embodiments, the access privilege setting is one of a plurality of selectable access privilege settings comprising access available only to: a creator of the notes application document, a group of selected user or a selected class of users, users with access to a categorized ultrasound topic, and/or users of a particular ultrasound machine 100. In certain embodiments, the display system 134 comprises a main display and a touch panel display. The notes application document having the received notes may be presented at a selected one or both of the main display and the touch panel display. The one or both of the main display and the touch panel display may comprise a dedicated area configured to present the notes application document and the notes application document having the received notes. In a representative embodiment, the plurality of data storage media 138 comprises a local archive 138, electronic medical records (EMR), and a Picture Archiving and Communication System (PACS). In an exemplary embodiment, the method 200 further comprises receiving 212, by the at least one processor 132, 140, a reminder time selection associated with the notes application document. The method 200 further comprises presenting 216, by the at least one processor 132, 140, a notification at the display system 134 at a time corresponding to the reminder time selection. The notification may provide access to the notes application document having the received notes.

Various embodiments provide an ultrasound system 100 for capturing categorized notes. The ultrasound system 100 may comprise at least one processor 132, 140 and a display system 134. The at least one processor 132, 140 may execute a notes application. The at least one processor 132, 140 may be configured to receive a selection to create a notes application document. The at least one processor 132, 140 may be configured to insert received notes in the notes application document. The at least one processor 132, 140 may be configured to assign one or both of an ultrasound topic category and an access privilege setting to the notes application document. The at least one processor 132, 140 may be configured to selectively store the notes application document having the received notes at one of a plurality of data storage media 138 communicatively coupled to the ultrasound system 100 based on the one or both of the ultrasound topic category and the access privilege setting assigned to the notes application document. The at least one processor 132, 140 may be configured to retrieve the notes application document having the received notes from the one of the plurality of data storage media 138. The display system 134 may be configured to present the notes application document in response to the selection to create the notes application document. The display system 134 may be configured to present the notes application document having the received notes retrieved from the one of the plurality of data storage media 138.

In an exemplary embodiment, the received notes inserted in the notes application document are separate and distinct from annotations embedded in an ultrasound image of an ultrasound examination. In various embodiments, the ultrasound topic category is one of a plurality of selectable ultrasound topic categories comprising an ultrasound machine 100, an ultrasound operator, a patient, and/or an ultrasound examination. In certain embodiments, the access privilege setting is one of a plurality of selectable access privilege settings comprising access available only to a creator of the notes application document, a group of selected user or a selected class of users, users with access to a categorized ultrasound topic, and/or users of a particular ultrasound machine. In a representative embodiment, the display system 134 comprises a main display and a touch panel display. The display system 134 may be configured to present the notes application document having the received notes at a selected one or both of the main display and the touch panel display. The one or both of the main display and the touch panel display may comprise a dedicated area configured to present the notes application document and the notes application document having the received notes. In an exemplary embodiment, the plurality of data storage media 138 comprises a local archive 138, electronic medical records (EMR), and a Picture Archiving and Communication System (PACS). In various embodiments, the at least one processor 132, 140 is configured to receive a reminder time selection associated with the notes application document. The at least one processor 132, 140 is configured to present a notification at the display system 134 at a time corresponding to the reminder time selection. The notification may provide access to the notes application document having the received notes.

Certain embodiments provide a non-transitory computer readable medium having stored thereon, a computer program having at least one code section. The at least one code section is executable by a machine for causing an ultrasound system 100 to perform steps 200. The steps 200 may comprise receiving 204 a selection to create a notes application document via a notes application executed at the ultrasound system 100. The steps 200 may comprise presenting the notes application document at a display system 134 of the ultrasound system 100. The steps 200 may comprise inserting received notes in the notes application document presented at the display system 134. The steps 200 may comprise assigning one or both of an ultrasound topic category and an access privilege setting to the notes application document. The steps 200 may comprise selectively storing 214 the notes application document having the received notes at one of a plurality of data storage media 138 communicatively coupled to the ultrasound system 100 based on the one or both of the ultrasound topic category and the access privilege setting assigned to the notes application document. The steps 200 may comprise retrieving 216 the notes application document having the received notes from the one of the plurality of data storage media 138. The steps 200 may comprise presenting 216 the notes application document having the received notes at the display system 134.

In various embodiments, the received notes inserted in the notes application document are separate and distinct from annotations embedded in an ultrasound image of an ultrasound examination. In certain embodiments, the ultrasound topic category is one of a plurality of selectable ultrasound topic categories comprising an ultrasound machine 100, an ultrasound operator, a patient, and/or an ultrasound examination. In a representative embodiment, the access privilege setting is one of a plurality of selectable access privilege settings comprising access available only to a creator of the notes application document, a group of selected user or a selected class of users, users with access to a categorized ultrasound topic, and/or users of a particular ultrasound machine 100. In an exemplary embodiment, the display system 134 comprises a main display and a touch panel display. The notes application document having the received notes may be presented at a selected one or both of the main display and the touch panel display. The one or both of the main display and the touch panel display may comprise a dedicated area configured to present the notes application document and the notes application document having the received notes. In various embodiments, the steps 200 may further comprise receiving 212 a reminder time selection associated with the notes application document. The steps 200 may further comprise presenting 216 a notification at the display system 134 at a time corresponding to the reminder time selection. The notification may provide access to the notes application document having the received notes.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for capturing categorized notes on an ultrasound system.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
receiving, by at least one processor of an ultrasound system executing a notes application, a selection to create a notes application document;
presenting, by the at least one processor via a display system of the ultrasound system, the notes application document;
inserting, by the at least one processor, received notes in the notes application document presented at the display system;
assigning, by the at least one processor, one or both of an ultrasound topic category and an access privilege setting to the notes application document;
selectively storing, by the at least one processor, the notes application document having the received notes at one of a plurality of data storage media communicatively coupled to the ultrasound system based on the one or both of the ultrasound topic category and the access privilege setting assigned to the notes application document;
retrieving, by the at least one processor, the notes application document having the received notes from the one of the plurality of data storage media; and
presenting, by the at least one processor, the notes application document having the received notes at the display system.

2. The method of claim 1, wherein the received notes inserted in the notes application document are separate and distinct from annotations embedded in an ultrasound image of an ultrasound examination.

3. The method of claim 1, wherein the ultrasound topic category is one of a plurality of selectable ultrasound topic categories comprising:
an ultrasound machine,
an ultrasound operator,
a patient, and/or
an ultrasound examination.

4. The method of claim 1, wherein the access privilege setting is one of a plurality of selectable access privilege settings comprising access available only to:
a creator of the notes application document,
a group of selected user or a selected class of users,
users with access to a categorized ultrasound topic, and/or
users of a particular ultrasound machine.

5. The method of claim 1, wherein:
the display system comprises a main display and a touch panel display,
the notes application document having the received notes is presented at a selected one or both of the main display and the touch panel display, and
one or both of the main display and the touch panel display comprise a dedicated area configured to present the notes application document and the notes application document having the received notes.

6. The method of claim 1, wherein the plurality of data storage media comprises:
a local archive,
electronic medical records (EMR), and
a Picture Archiving and Communication System (PACS).

7. The method of claim 1, further comprising:
receiving, by the at least one processor, a reminder time selection associated with the notes application document; and
presenting, by the at least one processor, a notification at the display system at a time corresponding to the reminder time selection, the notification providing access to the notes application document having the received notes.

8. An ultrasound system comprising:
at least one processor executing a notes application, the at least one processor configured to:
receive a selection to create a notes application document;
insert received notes in the notes application document;
assign one or both of an ultrasound topic category and an access privilege setting to the notes application document;
selectively store the notes application document having the received notes at one of a plurality of data storage media communicatively coupled to the ultrasound system based on the one or both of the ultrasound topic category and the access privilege setting assigned to the notes application document; and
retrieve the notes application document having the received notes from the one of the plurality of data storage media; and
a display system configured to:
present the notes application document in response to the selection to create the notes application document, and
present the notes application document having the received notes retrieved from the one of the plurality of data storage media.

9. The system of claim 8, wherein the received notes inserted in the notes application document are separate and distinct from annotations embedded in an ultrasound image of an ultrasound examination.

10. The system of claim 8, wherein the ultrasound topic category is one of a plurality of selectable ultrasound topic categories comprising:
an ultrasound machine,
an ultrasound operator,
a patient, and/or
an ultrasound examination.

11. The system of claim 8, wherein the access privilege setting is one of a plurality of selectable access privilege settings comprising access available only to:
a creator of the notes application document,
a group of selected user or a selected class of users,
users with access to a categorized ultrasound topic, and/or
users of a particular ultrasound machine.

12. The system of claim 8, wherein:
the display system comprises a main display and a touch panel display,
the display system is configured to present the notes application document having the received notes at a selected one or both of the main display and the touch panel display, and
one or both of the main display and the touch panel display comprise a dedicated area configured to present the notes application document and the notes application document having the received notes.

13. The system of claim 8, wherein the plurality of data storage media comprises:
a local archive,
electronic medical records (EMR), and
a Picture Archiving and Communication System (PACS).

14. The system of claim 8, wherein the at least one processor is configured to:
receive a reminder time selection associated with the notes application document; and
present a notification at the display system at a time corresponding to the reminder time selection, the notification providing access to the notes application document having the received notes.

15. A non-transitory computer readable medium having stored thereon, a computer program having at least one code section, the at least one code section being executable by a machine for causing an ultrasound system to perform steps comprising:
receiving a selection to create a notes application document via a notes application executed at the ultrasound system;
presenting the notes application document at a display system of the ultrasound system;
inserting received notes in the notes application document presented at the display system;
assigning one or both of an ultrasound topic category and an access privilege setting to the notes application document;
selectively storing the notes application document having the received notes at one of a plurality of data storage media communicatively coupled to the ultrasound system based on the one or both of the ultrasound topic category and the access privilege setting assigned to the notes application document;
retrieving the notes application document having the received notes from the one of the plurality of data storage media; and
presenting the notes application document having the received notes at the display system.

16. The non-transitory computer readable medium of claim 15, wherein the received notes inserted in the notes application document are separate and distinct from annotations embedded in an ultrasound image of an ultrasound examination.

17. The non-transitory computer readable medium of claim 15, wherein the ultrasound topic category is one of a plurality of selectable ultrasound topic categories comprising:
an ultrasound machine,
an ultrasound operator,
a patient, and/or
an ultrasound examination.

18. The non-transitory computer readable medium of claim 15, wherein the access privilege setting is one of a plurality of selectable access privilege settings comprising access available only to:
a creator of the notes application document,
a group of selected user or a selected class of users,
users with access to a categorized ultrasound topic, and/or
users of a particular ultrasound machine

19. The non-transitory computer readable medium of claim 15, wherein:
the display system comprises a main display and a touch panel display,
the notes application document having the received notes is presented at a selected one or both of the main display and the touch panel display, and
one or both of the main display and the touch panel display comprise a dedicated area configured to present the notes application document and the notes application document having the received notes.

20. The non-transitory computer readable medium of claim 15, further comprising:
receiving a reminder time selection associated with the notes application document; and
presenting a notification at the display system at a time corresponding to the reminder time selection, the notification providing access to the notes application document having the received notes.
